(19) United States
(12) Patent Application Publication
(10) Pub. No.: US 2025/0195601 A1
(43) Pub. Date: Jun. 19, 2025

(54) TURMERIC OIL ESSENTIAL-OIL BISABOLENE SESQUITERPENOIDS IN THE TREATMENT OF NON-NEURODEGENERATIVE DISEASES

(71) Applicant: UNIVERSIDAD DE CUENCA, Cuenca (EC)

(72) Inventors: Adriana Monserrath ORELLANA PAUCAR, Cuenca (EC); Enrique Santiago PERALTA PINTADO, Cuenca (EC)

(21) Appl. No.: 18/543,830

(22) Filed: Dec. 18, 2023

(51) Int. Cl. A61K 36/9066; A61P 21/00; A61P 25/00

(52) U.S. Cl. CPC A61K 36/9066; A61P 21/00; A61P 25/00

(57) ABSTRACT

The present invention relates to a method for treating non-neurodegenerative diseases, comprising administering to a subject in need thereof an effective amount of turmeric oil, wherein the oil is obtained from rhizomes of Curcuma longa L., and wherein the non-neurodegenerative diseases include upper motor neuron syndrome, lower motor neuron syndrome, spastic hemiparesis, tetraparesis, diparesis, monoparesis, flaccid hemiparesis, cerebral palsy, spasticity, autism, dysarthria, communication, speech, and language disorders.

### FIELD OF THE INVENTION

The present development relates to turmeric oil in the treatment of non-neurodegenerative diseases or disorders such as upper motor neuron syndrome, lower motor neuron syndrome, quadriparesis, diparesis, monoparesis, spastic hemiparesis, tetraparesis, flaccid hemiparesis, cerebral palsy, spasticity, autism, communication, speech, and language disorders, as well as executive functions impairment: metacognition impairment, visual fixation patterns impairment, and constipation.

### BACKGROUND OF THE INVENTION

Muscle tone is a physiological characteristic that represents the resting level of tension in a muscle, which prepares it for a rapid and reliable response to voluntary or reflexive commands. Tone is automatically generated by the impulse activity of the afferents that naturally excite alpha motoneurons, and it is controlled by four neural structures. First, neurons are in the ventral horn of the spinal cord grey matter. Second, neurons have cell bodies that lie in the brainstem and cerebral cortex and extend into the corticospinal tract. Third, the cerebellum exerts its control via the spinocerebellar tract. Last, the basal ganglia regulate the activities of the brainstem and cerebral cortex. Damage to interneurons, alpha moto neurons, and descending pathways causes changes in muscle tone. When muscle tone is decreased, joint stability is lost, and joint range of motion increases. Most flaccid hemiplegic patients show shoulder subluxation. Glenohumeral joint subluxation occurs when shoulder joint and shoulder girdle stability are lost. In addition, stroke patients with shoulder subluxation have decreased external shoulder rotation range of motion from the evaluation at stroke onset to six months after the stroke.

Likewise, in paraplegic patients with flaccid paralysis, hip dislocation quickly happens in passive hip adduction and extension. Muscle tone seems to influence the joint range of motion and stability (Hiengkaew, Vittayasoontorn, Pongporn, & Kaewtong, 2003).

Upper motor neuron syndrome refers to symptoms such as muscle weakness, decreased muscle control, easy fatigability, altered muscle tone, and exaggerated deep tendon reflexes (also known as spasticity), which can occur after a brain or spinal cord injury. The imbalance of muscle forces across a joint can lead to limb deformities that can be disfiguring, disabling, and painful (Mayo Clinic, 2023).

Motor neuron diseases (MNDs) are a group of progressive neurological disorders that destroy motor neurons, the cells that control skeletal muscle activity such as walking, breathing, speaking, and swallowing. Non-neurodegenerative diseases include post-polio syndrome. Messages or signals from nerve cells in the brain (upper motor neurons) are typically transmitted to nerve cells in the brain stem and spinal cord (lower motor neurons) and then to muscles in the body. Upper motor neurons direct the lower motor neurons to produce muscle movements. When the muscles cannot receive signals from the lower motor neurons, they weaken and shrink in size (muscle atrophy or wasting). The muscles may also start to twitch spontaneously. These twitches (fasciculations) can be seen and felt below the skin's surface. When the lower motor neurons cannot receive signals from the upper motor neurons, it can cause muscle stiffness (spasticity) and overactive reflexes. This can make voluntary movements slow and difficult. Over time, individuals with MNDs may lose the ability to walk or control other actions (National Institute of Neurological disorders and Strokes, 2023). Quadriparesis is defined as muscle weakness or partial paralysis in all four limbs, associated with a neurological injury or disorder called tetraparesis. Diparesis usually indicates that the legs are affected more than the arms, primarily affecting the lower body. Tetraparesis refers to weakness of all four limbs (National Library of Medicine, 2023).

Monoparesis (monoplegia) refers to partial (monoparesis) or complete (monoplegia) loss of voluntary motor function in a single limb. Cranial mononeuropathy implies that there is dysfunction referable to a single cranial nerve (Kornegay, 1991). It is also defined as weakness or paralysis of a limb, usually due to pathology of the spine and the proximal portion of nerves. Monoplegia may also be the initial presentation of hemiplegia, paraplegia, or quadriplegia (Piña-Garza, 2013).

Hemiparesis is the arm and leg weakness on the same side of the body. The face may or may not be involved. Hemiparesis may be mild to severe. It is almost always caused by lesions involving the corticospinal tract (DePiero, 2011). Spastic hemiplegia typically refers to unilateral paresis (weakness) with spasticity of the affected muscles and increased tendon reflexes (National Library of Medicine, 2023).

Cerebral Palsy (CP) refers to neurological disorders that appear in infancy or early childhood and permanently affect body movement and muscle coordination. CP is caused by damage to or abnormalities inside the developing brain that disrupt the brain's ability to control movement and maintain posture and balance. The term cerebral refers to the brain; palsy refers to the loss or impairment of motor function. Sometimes, the brain areas involved in muscle movement do not develop as expected during fetal growth. In others, the damage results from injury to the brain, either before, during, or after birth. In either case, damage is not reversible, and the resulting disabilities are permanent (National Institute of Neurological Disorders and Stroke).

Spasticity is a condition in which there is an abnormal increase in muscle tone or muscle stiffness, which might interfere with movement and speech or be associated with discomfort or pain. Spasticity is usually caused by damage to nerve pathways within the brain or spinal cord that control muscle movement (National Institute of Neurological disorders and Strokes, 2023).

Autism is a precocious behavioral constellation of social and communicative atypicalities associated with apparently restricted interests and repetitive behavior (RIRB) paired with an uneven ability profile. It is still considered a behavioral syndrome 75 years after its initial recognition by Kanner (1943) and under a different form by Asperger (1944). Kanner's and Asperger's views on autism were relatively clear-cut; their initial definition summarized the particularities presented in 11 children (4 for Asperger's) who were distinguishable from their peers. None of the children described by Kanner and Asperger had a recognizable neurologic, genetic syndrome in addition to autism. All had strong speech atypicalities and/or delay. None of these children were considered to be intellectually disabled, but their intelligence manifested itself in an atypical way. Autism was stable over time, though some developmental transformations were originally described and elaborated on in Kanner's follow-up papers. Although the autism category is historically centered on an easily recognizable clinical presentation, its extension, following accepted definitions, now encompasses a spectrum of presentations that vary in adaptive level, neurogenetic alterations, and similarity with the initial clinical image described by both Kanner and Asperger in the mid-20th century. The definition of autism is grounded on clusters of positive and negative clinical traits related to the social character and variety of overt behavior (symptoms), a situation that requires agreement among experts. In the DSM-5 (2013) categorical definition of “autism spectrum disorder,” socio-communicative symptoms should include quantitative or qualitative alteration in social communication and social interaction across multiple contexts, as manifested by deficits in social-emotional reciprocity and nonverbal communicative behaviors used for social interaction, as well as developing, maintaining, and understanding relationships. Furthermore, two symptoms of restricted, repetitive patterns of behavior, interests, or activities should be present among a list of four: stereotypical or repetitive motor movements, use of objects, or speech; insistence on sameness, inflexible adherence to routines, or ritualized patterns or verbal nonverbal behavior; highly restricted, fixated interests that are abnormal in intensity or focus; and hyper-or hyporeactivity to sensory input or unusual interests in sensory aspects of the environment. It encompasses the previous DSM-IV (1994) definition of autistic disorder, Asperger syndrome, and pervasive developmental disorders not otherwise specified. Symptoms must be present in the early developmental period and cause clinically significant impairment in present functioning. Intellectual disability is the only differential diagnosis when it can, by itself, explain a major part of the clinical presentation. The heterogeneity of autistic spectrum is intrinsic in its current definition, which reciprocally contributes to maintaining it (Mottron & Bzdock, 2020). While no autism subtypes are identified in DSM-5 (as Asperger syndrome was in DSM-IV, a form of autism without speech delay or impairment and excluding measured intellectual disability), four clinical specifiers are proposed: level of cognitive functioning, associated comorbid neurogenetic or psychiatric conditions, language level, and severity, identified as the level of assistance required for functioning. Each of these specifiers can be considered a quasi-dimensional variable, which results in extreme phenotypic variations, theoretically not interfering with the definite diagnosis. Intelligence can vary from the lowest levels, where a clinically significant distinction between autism and intellectual disability can be made, conventionally around 18 months of mental age, to the highest levels, where the clinical distinction between atypicality resulting from giftedness and autism may be challenging. Language level is a major factor of heterogeneity and is characterized by two subgroups in DSM-IV, with (autism) and without (Asperger) structural and/or developmental language atypicalities (Mottron, 2020). Autism is probably the only neurodevelopmental condition where speech can vary from inexistent to perfect. Comorbidity specifier comprises conditions associated with neurogenetics and psychiatry. The recognition of an inventory of diagnosable conditions associated with an autistic-like phenotype has resulted in the addition of another 10%-15% of individuals now described as showing syndromic, etiologic, or secondary autism, in contrast with idiopathic or primary autism where there are no identifiable syndromes apart from autism or only mutations that cannot be causally related to autism. Autistic people, particularly adults, also present identifiable psychiatric conditions that extend from calling the same condition another name (for instance, most autistic people being positive for the current criteria of avoidant personality disorder or speech disorder) to an actual, independent condition such as anxiety or mood disorder. Lastly, the severity specifier indicates that, aside from the mandatory presence of significant impairment, the level of adaptation may vary dramatically but must be at least “clinically significant” (Mottron, 2020).

Speech and language disorders refer to problems in communication and related areas, such as oral motor function.

Executive functions represent a constellation of cognitive abilities that drive goal-oriented behavior and are critical to adapt to an ever-changing world.

Executive functions can be split into four distinct components: working memory, inhibition, set shifting, and fluency. These components may be differentially affected in individual patients and act together to guide higher-order cognitive constructs such as planning and organization. Specific bedside and neuropsychological tests can be applied to evaluate components of executive function. While dysexecutive syndromes were first described in patients with frontal lesions, intact executive functioning relies on distributed neural network, including the prefrontal cortex and the parietal cortex, basal ganglia, thalamus, and cerebellum. Executive dysfunction arises from injury to any of these regions, their white matter connections, or neurotransmitter systems. Therefore, Dysexecutive symptoms occur in most neurodegenerative diseases and many other neurologic, psychiatric, and systemic illnesses. Management approaches are patient-specific and should focus on treating the underlying cause in parallel with maximizing patient function and safety via occupational therapy and rehabilitation (Rabinovici, Stephens, & Possin, 2015).

Metacognition reflects our capacity to monitor or evaluate other cognitive states unfolding during task performance, such as our confidence level in a memory's veracity. Impaired metacognition is seen in patients with traumatic brain injury (TBI) and substantially impacts their ability to manage functional difficulties during recovery. Recent evidence suggests that metacognitive representations reflect domain-specific processes (e.g., memory vs. perception) acting jointly with generic confidence signals mediated by widespread frontoparietal networks. The impact of neurological insult on metacognitive processes across different cognitive domains following TBI remains unknown (Fitzgerald, et al., 2022).

Constipation is a heterogeneous disorder that often is hypothesized to be associated with disordered movement through the large intestine, the anorectum, or both. Constipation has been associated with many diseases or as a side effect of many drugs. Diseases may produce constipation by directly impacting gastrointestinal function or result in physical or mental impairments that produce or exacerbate constipation. An example of the latter is physical immobility, which can lead to fecal retention. The most devastating disorders are neurologic diseases, which may directly affect the large intestine, produce generalized weakness or voluntary muscle dysfunction and lead to inanition or physical and emotional impairments (Arnold, 2004).

Turmeric (Curcuma longa L.) belongs to the Zingiberaceae family and is native to southeast India. Asians have used this old perennial plant for thousands of years. It is a major part of the Siddha system. This system has recommended the medicinal use of turmeric. Curcuminoids and essential oil from turmeric have shown various bioactivities and promising results in various scientific studies. Hence, the oil has been in high demand since ancient times and recently finds extensive application in flavor, perfumery, cosmetic, food products, beverages, and the pharmaceutical industry. This oil's nature and versatile applications highlight its importance in the global market, and India is one of the top commercial growers. Food and other industries recently focused on natural additives rather than chemicals due to health hazards. Therefore, turmeric is one of the most important alternatives because it is an abundantly and easily extractable, natural, safe oil used worldwide (Kuntal, 2016).

Patent U.S. Pat. No. 9,782,361 relates to the anticonvulsant activity of turmeric oil and its volatile bisabolene sesquiterpenoids ar-turmerone, α-turmerone, β-turmerone (curlone), and α-atlantone, as an anticonvulsant agent for the treatment of epilepsy and/or as a therapeutic agent for the treatment of disorders of the central nervous system, including tremor, pain, mood disorders (including depression, bipolar disorder, attention deficit-hyperactivity disorder, and schizophrenia), and neurodegenerative diseases.

US20210254158 provides methods for treating or ameliorating the effects of a disorder, such as schizophrenia or bipolar disorder, by increasing or decreasing proline levels. Further provided are methods of predicting and monitoring the clinical response in a patient and diagnostics systems for identifying a patient likely to benefit from proline modulation. Examples of SLC7A11, SLC1A3, SLC1A2, and SLC7A11 modulators that decrease proline levels include curcumin or analogs.

U.S. Pat. No. 10,512,616 relates to a formulation of curcuminoid with the essential oil of turmeric to enhance the bioavailability of curcumin and to augment the biological activity of curcumin, wherein the curcumin is the main constituent of curcuminoid and wherein ar-turmerone is the main constituent of the essential oil of turmeric.

### BRIEF DESCRIPTION

The present invention is directed to a method for treating non-neurodegenerative diseases, comprising administering to a subject in need thereof an effective amount of turmeric oil, wherein the oil is obtained from rhizomes of Curcuma longa L., and wherein the non-neurodegenerative diseases include upper motor neuron syndrome, lower motor neuron syndrome, spastic hemiparesis, tetraparesis, diparesis, monoparesis, flaccid hemiparesis, cerebral palsy, spasticity, autism, dysarthria, communication, speech, and language disorders.

### BRIEF DESCRIPTION OF THE FIGURES

FIG. 1. Aula Nesplora showing very low performance in attention span, vigilance, and processing speed (December 2018).

FIG. 2. Intellectual profile intelligence scale for adults (WAIS IV).

FIG. 3. Aula Nesplora test showing a significant correction in the deficiencies shown 2 years ago in attention, vigilance and processing speed (December 2020).

FIG. 4. Comparison of intellectual profile.

FIG. 5. Comparison of neuropsychological profile.

### DETAILED DESCRIPTION

Most pharmacological activities of turmeric have been explained by the properties of curcumin, mainly because turmeric oil has not been as extensively studied as curcuminoids. Turmeric rhizome oil (TO) is responsible for this spice's characteristic taste and smell. Dried rhizomes contain about 3-6% essential oil. The major TO constituents are sesquiterpenes: bisabolanes, guaianes, germacranes, caranes, elemanes, spironolactones, selinanes, santalanes, and caryophyllanes. Ar-turmerone, α-turmerone, and β-turmerone are the principal bisabolane sesquiterpenes. Other notable TO compounds with reported bioactivity are α-atlantone, ar-curcumene, γ-curcumene, eucalyptol, β-(Z)-farnesene, germacrone, β-curlone, p-cymene, z-citral, β-sesquiphellandrene, α-santalene, α-zingiberene, and l-zingiberene (Orellana-Paucar & Machado-Orellana, 2022).

The volatile oil of turmeric is light yellow and is responsible for the characteristic aroma of turmeric. Their most important components are mainly oxidized sesquiterpenes. Turmeric oil is extracted from ground turmeric root by steam distillation. With this method, the volatile oil released from the spice is transported along with the vapors and collected. The mixture of volatile oil and steam is then cooled in a condenser.

Additionally, turmeric oil is separated from water, collected for drying, and stored in a cool, dry place protected from light (Ravindran, Babu, and Sivaraman, 2007)

The term “essential oil,” “essential oil of turmeric,” or “turmeric oil” refers to “volatile oil” or “volatile oil of turmeric.” The essential oil is a mixture of oils obtained during the extraction of curcumin or curcuminoids from turmeric rhizomes. The volatile oil of turmeric is light yellow and is responsible for the characteristic aroma of turmeric. It is extracted from the rhizomes. Their most important components are mainly oxidized sesquiterpenes. Turmeric oil is primarily constituted of turmerones, α-turmerone (30-32%), β turmerone (15-18%), and ar-turmerone (dehydroturmerone) (17-26%) (Orellana-Paucar, A., 2020).

As used herein, the term “disorder” or “disease” broadly refers to a syndrome, condition, chronic illness, or a particular disease. For example, upper motor neuron syndrome, lower motor neuron syndrome, quadriparesis, diparesis, monoparesis, spastic hemiparesis, tetraparesis, flaccid hemiparesis, cerebral palsy, spasticity, autism, executive functions impairment: metacognition impairment, visual fixation patterns impairment, constipation and, communication, speech, and language disorders.

Other non-limiting examples of disorders, according to the present invention, include executive functions impairment, mental retardation, attention deficit, communication and language disorder, autism, attention deficit, impaired visual fixation, Pyramidal syndrome, upper motor neuron, dysparesis, spasticity, constipation, Guillain-Barré syndrome, flaccid tetraplegia.

As used herein, the terms “treat,” “treating,” “treatment” and grammatical variations thereof mean subjecting an individual subject to a protocol, regimen, or processor remedy in which it is desired to obtain a physiologic response or outcome in that subject, e.g., a patient. However, because every treated subject may not respond to a particular treatment protocol, regimen, or process, treating does not require that the desired physiologic response or outcome be achieved in each and every subject or subject population. Accordingly, a given subject or subject population may fail to respond or respond inadequately to treatment. The term “clinical response” as used herein, means a reduction of the severity or number of symptoms or characteristics of a disorder or disease during or following treatment.

The term “administering,” “administration” or variations thereof as used herein means introducing an agent, e.g., turmeric oil, into a subject's body, such as a human, in need of such treatment. The administration could be done by different routes of administration, e.g., orally.

In a present invention, an “effective amount” or “therapeutically effective amount” of turmeric oil is an amount of such material sufficient to effect beneficial or desired results as described herein when administered to a subject. Adequate dosage amounts will vary with the route of administration, the rate of excretion, the duration of the treatment, the identity of any other drug being administered, age, and size. In general, a suitable dose of the turmeric oil disclosed herein or a composition containing the same will be that amount of the active agent, e.g., turmeric oil, which is the lowest dose effective to produce the desired effect. In one embodiment, the dose is between 1-50 mg/kg/day of ar-turmerone. In an embodiment, the administration of turmeric oil is in a dose between 4 to 100 mg/day; in an additional embodiment, the administration of turmeric oil is in a dose between 9 to 30 mg/day.

### EXAMPLES

### Case 1: Executive Functions Impairment, Epilepsy, Mental Retardation, Attention Deficit

Female patient, without consanguinity of her parents, without significant prenatal or natal history. The mother describes normal psychomotor development. At two years of age, the patient presented seizures related to fever. At 11 years and six months old, she started with generalized tonic-clonic seizures upon awakening. The patient presented multiple ictal events until seizure control was achieved seven months later. However, up to 15 years old, the patient displayed sporadic events due to the inappropriate use of anticonvulsant drugs. Neuroimaging did not show structural alterations; the initial electroencephalogram showed bicentrotemporal spikes. The antiepileptic drugs 54597v1 10/28 administered were valproic acid and clobazam, which the patient maintained until date with occasional relapses due to irregular intake of the drugs.

At 15 years five months, in her first year of high school, she underwent her first psycho-pedagogical evaluation due to poor academic performance in all areas of study. The patient was assessed by applying the Wechsler Intelligence Scale for Children WISC-V (Wechsler & Corral, 2015) and NEUROPSI Attention and Memory (Ostrosky et al., 2014).

### First Evaluation

The patient appeared dressed and neat according to her age and gender. She demonstrated orientation, and her language was clear, sequential, and logical. There was clinical evidence of cognitive fatigue and slow processing, she required more time than expected to complete scheduled activities. The patient presented short attentional focus. It was possible to complete the evaluation process without behavioral maladjustments.

Intellectual profile: The patient possessed a total intelligence quotient (IQ) of 68 points, equivalent to “Mild Intellectual Development Disorder”. Thus, there was a 90% chance that her intellectual profile was between 64-75 (see Table 1).

TABLE 1
Main indices of the Wechsler Intelligence Scale for children.

| Main scales | PE | PC |
|---|---|---|
| Verbal comprehension (VCI) | 11 | 76 |
| Visuospatial (VSI) | 16 | 89 |
| Fluent Reasoning (FRI) | 9 | 69 |
| Working Memory (WMI) | 10 | 72 |
| Processing Speed (PS) | 10 | 72 |
| Full scale | 36 | 68 |

### Verbal Comprehension Index (VCI)

A composite score of 76 points was obtained, equivalent to below average performance. These tests allow an approach to the linguistic knowledge acquired for forming verbal concepts and reasoning.

### Visuospatial Index (VSI)

The patient achieved a composite score of 89 points, equivalent to a low average.

### Fluid Reasoning Index (FRI)

The patient obtained a composite score of 69, equivalent to very low.

### Working Memory Index (WMI)

The patient reached a composite score of 60 points, equivalent to very low.

### Processing Speed (PS)

Finally, the patient obtained a composite score of 68, equivalent to very low, reflecting slow cognitive speed.

Neuropsychological profile: Using the NEUROPSI II attention and memory test, a severe neuropsychological alteration was evidenced with a normalized score of 53 points (See Table 2).

TABLE 2
General neuropsychological index.

| TOTAL SCORES | NORMALIZED PT |
|---|---|
| Attention and executive | 77: slight alteration |
| functions | |
| Memory | 52: severe impairment |
| Memory and attention | 53: severe impairment |

### Attention

Results showed minor necessity for the attentional processes that imply the ability to focus and sustain attention, concentration, selective and sustained attention, and attentional control. In addition, the executive system score was also obtained. The executive system oversees the self-control capacity to handle complex cognitive and social situations and inhibition capacity. The patient reached a normalized score of 77, equivalent to mild alteration.

### Memory

The patient showed a performance with significant neediness in the tests evaluating the capacity of verbal and visual evocation in the short and long term and the coding processes. She obtained a normalized score of 52, equivalent to a severe alteration.

The Aula Nesplora test also displayed significant alterations in attentional capacity, impulse control, and hyperactivity.

Based on these results, prolonged-release methylphenidate was initiated, obtaining poor results. At 16 years and six months, due to her academic failure and her refusal to continue taking methylphenidate, it was decided to start using turmeric oil in a dosage of 36 mg/day.

The turmeric oil and two anticonvulsants (valproic acid and clobazam) were administered to the patient. Intermittent episodes of seizure relapse and remission were observed.

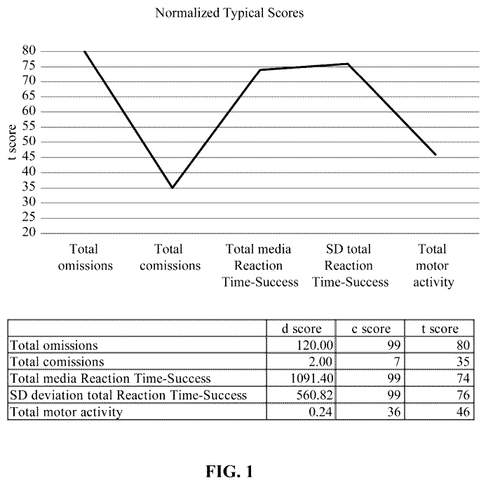

Nevertheless, a relevant improvement in behavior and school performance was clinically and academically noticeable (See FIG. 1).

### Second Evaluation

At 17 years and eight months, a second evaluation was carried out, considering the age of the patient. Thus, the WAIS IV test substituted the WISC V for adults (Wechsler, De la Guía, & Vallar, 2012), and the NEUROPSI Attention and Memory was applied again.

The patient presented herself dressed and neat according to her age and gender. The patient demonstrated self and allopsychic orientation, her language was clear, sequential, and logical. She was collaborative and quickly understood the instructions for each instrument. The time spent completing the activities oscillated within the parameters expected at her age. Her attentional focus was sustained.

### Intellectual Profile

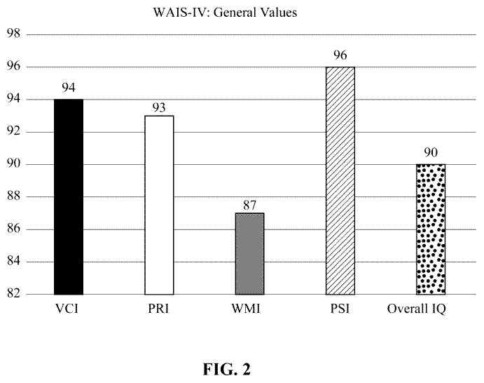

The patient obtained a total IQ of 90, corresponding to average intellectual potential. Thus, there was a 90% probability that her IQ corresponds to the values: 85-97. It is noteworthy that a favorable evolution was observed concerning the previous evaluation. She presented a preserved cognitive potential. Her scores dropped slightly in working memory (See FIG. 2).

Verbal Comprehension Index (VCI): The patient obtained a composite score of 94 points, equivalent to an average performance.

Perceptual Reasoning Index (PRI): The patient achieved an average composite score of 93 points.

Working memory index (WMI): The patient obtained a typical score of 87 points corresponding to low average concerning the one expected at her age.

Processing Speed Index (PSI): The patient achieved an average composite score of 96 points.

Neuropsychological profile: The results of applying the NEUROPSI Attention and Memory retest showed a neuropsychological performance with mild neuropsychological alteration and a normalized score of 83 points (See Table 3). A preserved profile was observed in the tests assessing her attention and executive system, while her memory maintains a slight alteration.

TABLE 3
Summary of the results obtained with
the second application of NEUROPSI

| TOTAL SCORES | NORMALIZED PT |
|---|---|
| Attention and executive | 93: average |
| functions | |
| Memory | 81: slight alteration |
| Memory and attention | 83: slight alteration |

Attention and concentration/Executive functions: The patient reached a normalized score of 93, according to the average, without significant alterations.

Memory: The patient presented a slight alteration. A performance with slight needs was observed in the tests that evaluate memory; the patient obtained a normalized score of 81, equivalent to a mild alteration.

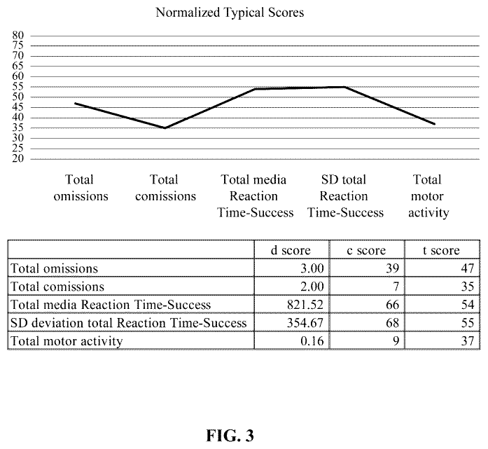

This favorable evolution was in line with the results obtained in the Aula Nesplora, which showed no alterations (see FIG. 3).

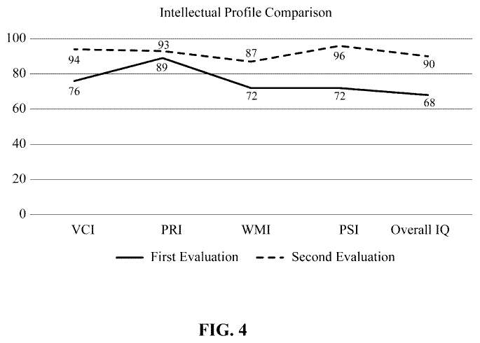

Analyzing the results of both the 2018 and 2020 evaluations, a notable evolution of the intellectual profile of the patient can be noticed, both in their total IQ reaching a composite score of 90, equivalent to average, with no significant alterations to mention. As can be seen in FIG. 4, academic performance in each of the evaluated indices improved considerably, except for the working memory scale, which increased, but not to the extent of the other measured areas.

These results agreed with the improvement observed in the neuropsychological evaluation, in which she showed significant progress in her maturity index, from a normalized score of 52 (severe alteration) in the first assessment to a total score of 83 on the retest. Thus, it was concluded that the patient has matured in her neuropsychological profile, achieving an index within the average in the tests that evaluate her attention and executive system. At the same time, a slight alteration remained in her memory area.

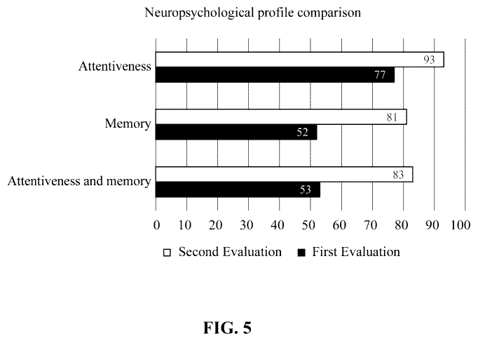

Conversely, a cognitive enrichment in the patient's profile was evident. This enhancement was reflected in her academic performance and her general life development. The patient presented seizures relapse and maintained antiepileptic drugs administration. Noteworthy, while using turmeric oil, the patient was no longer required to use methylphenidate (See FIGS. 4 and 5).

### Case 2: Communication and Language Disorder, Autism, Attention Deficit, Impaired Visual Fixation

Female patient of two years and three months old, daughter of non-consanguineous parents, with no significant prenatal or natal history. The parents attended the medical consultation because the patient did not respond to her name and did not understand or obey orders. The patient did not speak. She was extremely restless and had constant fluttering and no visual fixation. Melatonin was prescribed because the patient woke up often at night.

An ADI-R and ADOS-2 test was applied obtaining highly summarized results, expressing moderate-to-severe concern and a score greater than 24. The neurological examination displayed no sensory, motor, or cranial nerve alterations. The patient's hearing assessment was normal. The concomitant administration of turmeric oil and CBD was initiated.

The parents reported that the patient was very irritable and verbally repeating her desire to avoid medication. Six months following the concomitant administration of turmeric oil and CBD, she displayed at least 30 words in her language, was able to form sentences, to understand commands, and to communicate her needs. The dosage was as follows: 14.4 mg/day of turmeric oil and 5 mg B.I.D. of CBD.

### First Evaluation

The patient attended dressed and clean, according to her age and gender. After initial play activities she joined the activities with an exploratory game. She achieved functional play, was able to hold her gaze for short periods, her attentional focus was sustained, and she showed proper motor activity. The patient explored the room and objects and integrated them into a functional game with dolls and kitchen toys. At first, the patient was shy, but she managed to adapt. The patient did not require the mother's presence to stay in the evaluation room. She lasted in the workroom for 40 minutes, with pauses of 10 minutes.

Interactive competence: In the games, the patient showed communication with her mother, pointed at objects that she liked, and participated in the proposed activities. The patient could maintain eye contact for short periods.

Communicative aspects: When the patient wanted a specific object and she could not get it by herself, she had difficulty asking for it.

Spontaneous verbal emissions: The patient emitted single words and short phrases with articulatory difficulties. Sometimes, she repeated words mentioned by other people.

Comprehensive language: The patient responded to her name without difficulty and understood and executed commands after modeling. In the case of verbal instructions, these must be repeated several times. In addition, the patient presented sporadic echolalia and a flat tone during the evaluation sessions.

PSYCHO-EDUCATIONAL PROFILE
WECHSLER intelligence scale for preschool
and elementary school WIPPSI IV

| Test | PD | | | | | | |
|---|---|---|---|---|---|---|---|
| Cubes | 12 | 7 | | 7 | | | |
| Information | 10 | 4 | 4 | | | | |
| Arrays | 14 | 14 | | | 14 | | |
| Animal search | 10 | 6 | | | | | 6 |
| Recognition | 10 | 7 | | | | 7 | |
| similarities | 6 | 6 | 6 | | | | |
| Concepts | 6 | 8 | | | 8 | | |
| Cancellation | 8 | 4 | | | | | 4 |
| Location | 7 | 7 | | | | 7 | |
| Puzzle | 14 | 8 | | 8 | | | |
| Sum Point Scalars | | 44 | 10 | 15 | 22 | 14 | 10 |
| | | | VCI | VSI | FRI | WMI | PSI |

| SCALE | | Scalar Score | CI |
|---|---|---|---|
| Verbal | VCI | 10 | 72 |
| Comprehension | | | |
| Index | | | |
| Visuospatial Index | VSI | 15 | 85 |
| Fluid Reasoning | FRI | 22 | 106 |
| Index | | | |
| Working | WMI | 14 | 81 |
| Memory Index | | | |
| Processing | PSI | 10 | 73 |
| Speed Index | | | |
| FULL SCALE | | 44 | 79 |

### Reference Mean: 90/109

Cognitive potential: The obtained results suggested that the patient has a total intelligence quotient of 79 points, equivalent to low intellectual capacity. Thus, there was a 95% chance that her total IQ is between 74-87, with verbal comprehension and processing speed needs. She displayed a significant strength in fluid reasoning.

Verbal comprehension index (VCI): (72 low). Low performance was observed concerning the average in the measure of knowledge acquired from the environment, the formation of verbal concepts, and verbal reasoning of the patient. It is noteworthy that, in this test, the patient must give oral answers to all the items applied.

Visuo-Spatial Index (VSI): (85 medium-low). A low average performance was observed in the visuo-spatial processing measure, the integration and synthesis of relations part, the attention to visual details, the formation of non-verbal concepts, and visuomotor integration. It is a measure that requires the manipulative IQ.

Fluid Reasoning Index (FRI): (106 medium). An average performance was observed in their fluid reasoning, ability to detect underlying notional relationships between visual objects, and using reasoning to identify and apply rules. Inductive and quantitative reasoning, general visual intelligence, and simultaneous processing were evaluated.

Working Memory Index (WMI): (81 medium-low). It measures visual working memory, visuospatial working memory, and the ability to show resistance to interference. This scale evaluates attention and concentration, mental control, and reasoning.

Processing Speed Index (PSI): (73 low). A low score relative to average performance was observed in the ability to explore, order, or discriminate simple visual information. This scale also measures short-term visual memory, visuomotor coordination, cognitive flexibility, and test-taking speed.

### Discrepancy Analysis:

A strong point in fluid reasoning was observed within the Table of comparisons between the scores obtained by the patient whereas that a weak point in verbal comprehension and processing speed was also observed.

CUMANIN Child Neuropsychological Maturity Questionnaire

| SCALE | PERCENTILE | EQUIVALENCE |
|---|---|---|
| PSYCHOMOTOR | 30 | MEDIUM-LOW |
| ARTICULATORY | 5 | VERY LOW |
| LANGUAGE | | |
| LANGUAGE EXPRESSIVE | 15 | LOW |
| COMPREHENSIVE | 5 | VERY LOW |
| LANGUAGE | | |
| SPACE | 5 | VERY LOW |
| VISOPERCEPTION | 10 | LOW |
| ICONIC MEMORY | 25 | MID LOW |
| RHYTHM | 30 | MEDIUM |
| ATTENTION | 5 | VERY LOW |
| CD | LESS THAN 65 | VERY LOW |

From these results, it was inferred that the patient has a global neuropsychological development quotient with a percentile of less than 65 (very low) concerning the average expected for her age. In conclusion, there was a delay in development with a significant affectation of the verbal area (expression, comprehension, and articulation), visual motor skills, and attention.

TEPSI PSYCHOMOTOR TEST

| SUBTEST | CAT DESCRIPTIVE | P. EXPECTED |
|---|---|---|
| COORDINATION | 24 delay | 40-50 |
| LANGUAGE | 39 risk | 40-50 |
| MOTOR | 36 risk | 40-50 |
| TOTAL SCORE | 31 risk | 40-50 |

The results suggested the patient was within the risk zone for delay in language development, psychomotor skills, and coordination.

### Basic Skills Assessment

The Patient could:
Identify parts of her body.
Identify spatial notions: above, below, next to, behind, near.
Jump with feet together and stand on one foot for 5 seconds.
Draw a straight line.
Identify the onomatopoeic sound of animals.
Identify primary and secondary colors.
Count to 10.
Identify the vowels: a, i, o, u.
Identify basic geometric figures: circle, square, triangle.
Build a 4-piece puzzle.

### Drawing of the Human Figure:

### The Patient had an Evolutionary Age of 3 Years 0 Months.

### Specialized Assessment of Autism

### Observational Scale for the Diagnosis of Autism ADOS 2

Selected module: Module 1—Algorithm of some words
ADOS 2 is a semi-structured and standardized instrument to obtain information from the areas of reciprocal social interaction and repetitive behavior, restricted to determine the clinical diagnosis of autism spectrum disorder (ASD). It includes play-based activities such as bubble play, releasing an inflated balloon, imitation activities, pretend birthday parties, and free-play opportunities.

| LUATED AREAS | TOTAL |
|---|---|
| SOCIAL IMPACT | 7 |
| RESTRICTED BEHAVIOR | 1 |
| TOTAL | 8 |

EQUIVALENCE FOR DIAGNOSIS:

| SCORE | EQUIVALENCE |
|---|---|
| LESS THAN 5 YEARS | |
| 12 OR MORE | AUTISM |
| 8-11 | AUTISM SPECTRUM |
| LESS THAN 7 | NO ASD |

The patient's mother was present during the application of the test. The patient managed to articulate single words and 3-word phrases with significant difficulty. She presented an exploratory and symbolic game. The patient mainly entertained herself by exploring the toys. She played representative games of caring for and feeding animals.

As can be seen in the results matrix, the patient obtained a score of 7 in the domain of social affectation (SA) and 1 in the domain of repetitive restricted behavior (RRB). Thus, from these results it can be inferred that the patient exhibited mild features that correspond to the autism spectrum.

The patient possessed adequate motor activity. Prior to starting the test application, the patient was shy, greeted people, collaborated with the proposed activities, and managed to maintain social interaction with difficulty. The patient frequently interacted mainly with her mother, to whom she frequently showed the toys. The patient managed to keep her attention for short periods (5-10 consecutive minutes) and played exploratory games. During short periods, shared enjoyment was achieved in social interaction activities such as bubbles. She focused her attention on the proposed activities for brief periods. Activities involving imitation were accomplished with difficulty and eye contact was occasional.

The patient showed difficulty in imitation activities. The patient completed the anticipation activity of a routine with objects.

Regarding free play, the patient performed exploratory play. She represented the baby and animal care; she played feeding them and reading them stories. The patient showed a special interest in the phone toy.

In response to name activities, the patient responded to the call of her name without difficulty. However, she struggled in activities requiring shared attention.

During the application of the tests, the patient did not have tantrums. The quality of the relationship with the evaluator was good. The patient presented recurring difficulties asking for what she wanted. The patient showed sporadic immediate echolalia. She participated in the birthday party and collaborated without difficulty.

Social Communication Questionnaire SCQ

| | CUT-OFF | |
|---|---|---|
| SCORE | POINT FOR | |
| OBTAINED | DIAGNOSIS | DESCRIPTIVE CATEGORY |
| 15 | 15 | The patient presented mild features |
| | | compatible with the autism spectrum. |
| | | To a large extent, behaviors have |
| | | evolved favorably. |

Results obtained through the interview with the patient's mother allowed concluding that the patient presented features that are within the autism spectrum, mild degree. It is worth mentioning that the behavior associated with ASD showed a favorable evolution in relation to the description of the mother.

Patient behavior associated with ASD is described below:

| | | STEREOTYPICAL, |
|---|---|---|
| SOCIAL INTERACTION | COMMUNICATION | RESTRICTED AND |
| PROBLEMS | DIFFICULTIES | REPETITIVE BEHAVIOR |
| 1. Using other people's | 1. Difficulty in | 1. The repetitive use of objects |
| bodies to communicate was | developing a | improved. |
| overcome. | conversation was | 2. Weird sensory interests were |
| 2. Difficulty in | present. | present. |
| maintaining a direct gaze | 2. Stereotyped | 3. Hypersensitivity to certain |
| improved. | expressions were | sounds was present. |
| 3. Difficulty in making | present. | |
| friends was present. | 3. Confusion of | |
| 4. Difficulty in social | pronouns was present. | |
| interaction was present. | 4. Verbal routines were | |
| 5. Problems in make- | present. | |
| believe and group games was | | |
| present. | | |

### Development Evaluation

The Batelle Development Inventory was applied to the patient. This is an instrument for assessing developing skills and diagnosing possible deficiencies in different areas. The following results were obtained:

| | | Age of development of | | Age of |
|---|---|---|---|---|
| | Area | subareas | | development |
| Personal-Social | | | | 39 months |
| Adaptative | | | | 48 months |
| | Motor | Gross Motor | 49 months | 42 months |
| | | Fine Motor | 36 months | |
| | Communication | Receptive | 46 months | 40 months |
| | | Expressive | 36 months | |

| | |
|---|---|
| Cognitive | 43 months |
| Overall | 42 months |

Cognitive test: Patient was able to assemble 6-piece puzzle in 1 minute. She matched circle, square, and triangle on a stringing board, skewered 9 figures out of 9 on a board, skewered 6 pegs on a board in 10 seconds, screwed and unscrewed jars, matched pictures, imitated actions, counted to 10, recognized numbers and sizes, classified by size and weight. In addition, she understood the concept of “quantity,” but not the concept of “more” or the number-quantity relationship. She did not complete analogies.

Language test: The patient answered questions, although she repeated the question. She understood the use of objects and their functionality. The patient recognized simple negations, plural forms, appropriately answered yes/no questions, and elaborated 5-word sentences.

Motor test: The patient grabbed the pencil with intermediate grip (tripod), copied a circle, vertical and horizontal strokes, built towers of 8 cubes, slightly screwed. She did not imitate square and triangle forms or V, T, X strokes. She was not able to cut with scissors following a line of 10 cm. She walked up and down the stairs alternating feet, balanced on one foot, kicked a ball, threw the ball with direction, ran, and jumped.

Personal/social test: In absence of her mother, the patient related to the examiner, asked for help, responded to orders, and followed instructions. The patient played next to a child, but she did not interact with him or other children. If they tried to greet her or shake hands, the patient hid behind the adult in charge.

Adaptive Testing: The patient paid attention, completed a task without supervision, stayed seated even when there was no adult present. She used a spoon to eat. She slightly removed her clothes and tried to put them on. Also, she was able to notify her need to go to the toilet, when needed.

### Conclusion:

The diagnose was a global development delay with moderate affectation of fine psychomotor development and articulatory and comprehensive and expressive language.
The results suggested that the patient had a total IQ of 79 points, equivalent to low. There was a 95% chance that her total IQ is between 74-87.
The patient presented needs in verbal comprehension and processing speed. She displayed a significant strength in fluid reasoning.
The patient showed traits that are within the autism spectrum. Traits were in remission.
The patient had qualitative alterations in communication and social interaction.
Her psychomotor developmental age corresponded to 3 years and 0 months.

### Development Profile

Chronological age: 49 months (4 years and 1 month)
Age of global development: 42 months
Cognitive age: 43 months
Age in the adaptive area: 48 months Global motor age: 42 months
Age-gross motor: 49 months
Fine motor age: 36 months
Age in the personal/social area: 39 months.
Mild delay in cognitive and personal/social development
Moderate delay in fine motor skills
Evolution:

The patient approved the first grade without academic problems and without any adaptation in the curriculum. She still showed some communication problems, although she was able to make new friends on her own initiative.

### Case 3: Pyramidal Syndrome, Upper Motor Neuron, Dysparesis, Spasticity, Constipation

Male patient of two years and two months age. During an inguinal hernia surgery, he presented indefinite cardiorespiratory arrest that led to severe hypoxic-ischemic encephalopathy with cortico-subcortical cerebral atrophy. Because of this event, the patient developed spastic diplegia, neurodevelopmental regression, and structural focal epilepsy.

One year later, the patient had no neck control, and he was in the process of freeing the thumb. Development assessment reported an age of 30 months. In addition, he presented constipation. Pharmacological therapy included valproic acid and baclofen for seizure control and muscle tone improvement, respectively.

Four months later and one year after the hypoxic event, the recovery was modest with valproic acid and baclofen administration. The patient showed no neck control, tended to drowsiness, fixed gaze, and appeared to understand some orders. Constipation persisted. Administration of turmeric oil was started with a dose of 9 mg per day.

Seven months later, the patient displayed complete head control and axial hypotonia with better trunk control without reaching a sitting position. He showed improved fine motor skills and language. Also, the patient showed complete contact with the environment and was in the process of recognizing figures. Four months later, the administration of valproic acid and baclofen was suspended. Only turmeric oil was administered.

In a follow-up control, axial hypotonia and peripheral hypertonia were considerably reduced. The patient could walk with the support of two hands and was able to ride a bicycle with support wheels.

The patient was capable of reading and writing with certain difficulty in Spanish and English. He was studying in a regular school and still used a wheelchair due to inability of walking autonomously.

### Case 4. Mixed Cerebral Palsy: Pyramidal and Extrapyramidal, Epilepsy, Constipation, Mental Retardation

Female patient of four years and nine months of age. She was the daughter of non-consanguineous parents, born of a twin pregnancy by cesarean section at 30 weeks' gestation. The patient had multiple complications in her neonatal period with prolonged mechanical ventilation, including neuro-infection and neonatal seizures.

During the medical evaluation, the patient did not show neck control, sitting position, or language development. Central hypotonia and peripheral hypertonia, dystonia and dyskinesia, ROT++++/++, bilateral plantar flexor reflex were present.

One year later, a simple brain tomography showed diffuse cortical, subcortical atrophy, with areas of gliosis in the frontal region and greater ex-vacuo ventricular dilatation on the right. No advances regarding her neurological condition were identified. The pharmacological therapy included baclofen to improve muscle tone and levetiracetam to control seizures.

At eight years of age, the seizures were not controlled, and oxcarbazepine was added to her treatment with baclofen and levetiracetam. Three and a half years later, the patient showed no neck control and the peripheral tone decreased. Scoliosis, retractions, and joint deformities were still visible. Dystonias and dyskinesias were persistent.

In the subsequent 7.5 years, the patient had epileptic seizures of irregular onset and insufficient advances in her motor development. Administration of turmeric oil began with a dose of 27 mg per day.

In the next three years, the patient showed scoliosis without progression and a complete neck control. She improved hypertonia and a significant decrease in dystonia and dyskinesia was observed. The patient interacted appropriately with the environment, fixed her gaze, followed objects, and pronounced isolated words.

### Case 5. Guillain-Barré Syndrome, Flaccid Tetraplegia

Female patient of ten years and five months. She was the daughter of non-consanguineous parents. Parents referred that one day the patient woke up but could not walk. Noteworthy, the day before this event, she received a COVID vaccine. The patient was taken to a hospital and evaluated in the emergency room. Demyelinating motor polyradiculoneuropathy of severe degree with distal predominance, more significant in lower extremities was reported after performing electromyography and nerve conduction velocity analysis. On the third day at the hospital, the patient lost walking, sitting, neck control and lower and upper extremities mobility. No mechanical ventilation was required.

Treatment was based on gamma globulin, and she was discharged after three weeks of hospitalization with no improvement in her condition, with flaccid, areflexic tetraplegia, with no mobility in the lower extremities (0/5) and scant mobility in the upper extremities (1/5). Thus, the patient was prostrate, and it was recommended to start administering turmeric oil in a dose of 21.6 mg per day.

Four months later, the patient sat with difficulty. She presented poor movement in the inner extremities (1/5) and upper extremities (2/5) and generalized areflexia. The following three months, the patient started walking with support, with strength 2-3/5 in the upper extremities and 1-2/5 in the lower extremities. Generalized areflexia was still present.

After two more months, the patient presented limited independent walking with the support of a walker. Strength improved in upper extremities (proximal 3-4/5, distal 2/5), ROT++/++ was found. Likewise in the lower extremities (proximal 2-3/5, distal 1-2/5). Areflexia was also observed.

After an additional period of six months, the patient stood unassisted and showed an independent gait without support. Proximal strength was 4/5 and distal was 3/5 in the upper extremities, ROT++/++. In the lower extremities, proximal strength was 3/5 and distal was 2/5, Achillean ROT+/++, and patellar areflexia.

What is claimed is:

1. A method for treating non-neurodegenerative diseases, comprising administering an effective amount of turmeric oil to a subject in need thereof.

2. The method according to claim 1, wherein the turmeric oil is obtained from Curcuma longa rhizomes.

3. The method according to claim 1, wherein the non-neurodegenerative diseases are selected from upper motor neuron syndrome, lower motor neuron syndrome, spastic hemiparesis, tetraparesis, diparesis, monoparesis, flaccid hemiparesis, cerebral palsy, spasticity, autism, dysarthria, communication, speech, and language disorders.

4. A turmeric oil for use in non-neurodegenerative diseases.

5. The turmeric oil for use in non-neurodegenerative diseases according to claim 4, wherein the non-neurodegenerative diseases are selected from upper motor neuron syndrome, lower motor neuron syndrome, quadriparesis, diparesis, monoparesis, spastic hemiparesis, tetraparesis, flaccid hemiparesis, cerebral palsy, spasticity, autism, communication speech, and language disorders.

6. Use of turmeric oil in the treatment of non-neurodegenerative diseases.

7. The use according to claim 6, wherein the non-neurodegenerative diseases are selected from upper motor neuron syndrome, lower motor neuron syndrome, quadriparesis, diparesis, monoparesis, spastic hemiparesis, tetraparesis, flaccid hemiparesis, cerebral palsy, spasticity, autism, communication speech, and language disorders.
